(19) United States
(12) Patent Application Publication
(10) Pub. No.: US 2003/0162758 A1
(43) Pub. Date: Aug. 28, 2003

(54) Treatment for age-related macular degeneration (AMD)

(76) Inventors: Daniel M. Schwartz, San Francisco, CA (US); Keith Duncan, San Francisco, CA (US); Kathy Bailey, San Francisco, CA (US); John Kane, San Francisco, CA (US); Brian Ishida, Walnut Creek, CA (US)

Correspondence Address: FULBRIGHT & JAWORSKI, LLP, 1301 MCKINNEY, SUITE 5100, HOUSTON, TX, 77010-3095, US

(21) Appl. No.: 10/313,641

(22) Filed: Dec. 6, 2002

Related U.S. Application Data
(60) Provisional application No. 60/340,498, filed on Dec. 7, 2001.
(60) Provisional application No. 60/415,864, filed on Oct. 3, 2002.

(51) Int. Cl. A61K 31/58; A61K 31/56; A61K 31/381; A61K 31/203; A61K 31/192

(52) U.S. Cl. 514/172; 514/178; 514/567; 514/559; 514/438; 514/570

(57) ABSTRACT

The present invention addresses the treatment of age-related macular degeneration using regulation of pathogenic mechanisms similar to atherosclerosis. In further specific embodiments, reverse cholesterol transport components, such as transporters and HDL fractions, are utilized as diagnostic and therapeutic targets for age-related macular degeneration. In a specific embodiment, the lipid content of the retinal pigment epithelium, and/or Bruch's membrane is reduced.

### CROSS-REFERENCE TO RELATED APPLICATIONS

The present invention claims priority to U.S. Provisional Patent Application No. 60/340,498, filed Dec. 7, 2001 and U.S. Provisional Patent Application No. 60/415,864, filed Oct. 3, 2002, both of which are incorporated by reference herein in their entirety.

### FIELD OF THE INVENTION

The present invention is directed to the fields of ophthalmology and cell biology. Specifically, the invention regards treatment of age-related macular degeneration (AMD) utilizing regulation of reverse cholesterol transport.

### BACKGROUND OF THE INVENTION

Age-related macular degeneration (AMD) is the leading cause of severe visual loss in the developed world (Taylor et al., 2001; VanNewkirk et al., 2000). In the early stages of the disease, before visual loss occurs from choroidal neovascularization, there is progressive accumulation of lipids in Bruch's membrane (Pauleikhoff et al., 1990; Holz et al., 1994; Sheraidah et al., 1993; Spaide et al., 1999). Bruch's membrane lies at the critical juncture between the outer retina and its blood supply, the choriocapillaris. Lipid deposition causes reduced hydraulic conductivity and macromolecular permeability in Bruch's membrane and is thought to impair retinal metabolism (Moore et al., 1995; Pauleikhoff et al., 1990; Starita et al., 1996). Retina and/or RPE may respond by elaboration of angiogenic factors (e.g. VEGF, vFGF) that promote growth of choroidal neovascularization.

Interestingly, lipid accumulation in Bruch's membrane similar to that in AMD has been observed in apolipoprotein E (apo E) null mice (Dithmar et al., 2000; Kliffen et al., 2000). Because of the additional association between apo E alleles and other age-related degenerations, Alzheimer's disease and atherosclerosis, there has been recent investigation into a potential role for apo E in AMD.

Several studies on apo E polymorphism in AMD have been conducted (Simonelli et al., 2001; Klayer et al., 1998; Souied et al., 1998). In contrast to Alzheimer's disease, the apo E-4 allele has been associated with reduced prevalence of AMD. Apo E-2 allele is slightly increased in patients with AMD. Further supporting a role in AMD pathogenesis, apo E has been detected in drusen, the Bruch's membrane deposits that are the hallmark of AMD (Klayer et al., 1998; Anderson et al., 2001). Immunohistochemistry on post-mortem eyes has demonstrated apo E in the basal aspect of the retinal pigment epithelium (RPE) (Anderson et al., 2001). Cultured RPE cells synthesize high levels of apo E mRNA, comparable to levels found in brain (Anderson et al., 2001).

While the role of apo E in AMD is not established, this apolipoprotein has several functions that may affect the course of this disease. Apo E has anti-angiogenic (Browning et al., 1994), anti-inflammatory (Michael et al., 1994), and anti-oxidative effects (Tangirala et al., 2001). These are all considered atheroprotective attributes of Apo E, but may also be important in protecting against progression of AMD. While atheroprotective effects of apo E were initially thought to stem from effects on plasma lipid levels, local effects on vascular macrophages are probably equally important. Thus, selective enhanced expression of macrophage apo E in the arterial wall reduces atherosclerosis in spite of hyperlipidemia (Shimano et al., 1995; Bellosta et al., 1995; Hasty et al., 1999). Conversely, reconstitution of apo E null macrophages in C57BL/6 wild type mice induces atherosclerosis (Fazio et al., 1994). Atheroprotective effects of arterial apo E expression are thought to derive in part from facilitation of reverse cholesterol transport (Mazzone et al., 1992; Lin et al., 1999). The mechanisms by which apo E facilitates reverse cholesterol transport are incompletely understood. Apo E expression increases cholesterol efflux to HDL3 in J774 macrophages (Mazzone and Reardon, 1994) and lipid free apolipoprotein A1 (Langer et al., 2000). Cell surface apo E is also hypothesized to induce efflux from the plasma membrane (Lin et al., 1999).

Reverse cholesterol transport may be important in the pathogenesis of AMD because of lipid efflux from RPE into Bruch's membrane. Very much like intimal macrophages, RPE cells progressively accumulate lipid deposits throughout life; however, unlike vessel wall macrophages, the source of RPE lipid is thought to be retinal photoreceptor outer segments (POS) (Kennedy et al., 1995). Every day, each RPE cell phagocytoses and degrades more than one thousand POS via lyzosmal enzymes. These POS are enriched in phospholipid and contain the photoreactive pigment, rhodopsin. Incompletely digested POS accumulate as lipofuscin in RPE. By age 80, approximately 20% of RPE cell volume is occupied by lipofuscin (Feeney-Burns et al., 1984).

Analysis of Bruch's membrane lipid reveals an age-related accumulation of phospholipid, triglyceride, cholesterol, and cholesterol ester (Holz et al., 1994; Curcio et al., 2001). The origin of these lipids also is thought to derive principally from POS rather than from the circulation (Holz et al., 1994; Spaide et al., 1999). POS lipids are hypothesized to efflux from the RPE into Bruch's membrane. Although cholesterol ester deposition in Bruch's suggests contribution from plasma lipids, biochemical analysis of these esters suggests esterification of intracellular cholesterol by RPE cell derived ACAT (Curcio et al., 2002). While trafficking of lipids from the retina to RPE cells has been studied extensively, mechanisms of lipid efflux from RPE to Bruch's membrane are not well understood. Furthermore, from a pathogenic standpoint, regulation of lipid efflux into Bruch's membrane may be important in determining the rate of lipid-induced thickening that occurs in aging.

Nuclear hormone receptor ligands regulate reverse cholesterol transport in macrophages via their effects on ABCA-1 and apo E expression. Liver X receptor (LXR) and/or retinoid X receptor (RXR) ligands increase levels of these transporters and increase reverse cholesterol transport in macrophages (Mak et al., 2002; Laffitte et al., 2001). Thyroid hormone has also been demonstrated to increase expression of apo E three fold in HepG2 cells (Laffitte et al., 1994).

In AS, similar to AMD, lipids accumulate in the extracellular matrix and within phagocytic cells, primarily macrophages. Mechanisms of lipid metabolism in AS have been investigated in detail. Similar investigations into lipid processing by RPE and subsequent lipid efflux into BM and the circulation have not been conducted with the same depth as those for AS. As a consequence, potential therapeutic approaches to dry AMD are wonting.

Mullins et al. (2000) describe compositional similarity between drusen and other extracellular deposits, including atherosclerotic plaques. Specifically, vitronectin, amyloid P, Apo E, and lipids are among the constituents shared in common. More specifically, apolipoprotein E is identified in retinal pigmented epithelium.

Friedman (2000) reviews the role of atherosclerosis in the pathogenesis of AMD. Specifically, the review mentions targeting the angiogenesis pathway for treating the neovascular form of AMD, such as the member VEGF. It is noted that interfering with the upregulation or action of angiogenic agents may prove helpful for choroidal neovascularization, and, in alternative embodiments, statins may be useful for lowering the risk of AMD.

Anderson et al. (2001) reports apolipoprotein E protein is found in the same location as drusen, likely originating from the retinal pigmented epithelium.

U.S. Pat. No. 6,071,924 regards inhibition of proliferation of retinal pigment epithelium by contacting RPE cells with a retinoic acid receptor agonist, except for retinoic acid, preferably thereby inhibiting AP-1-dependent gene expression. In specific embodiments, an AP1 antagonist is delivered to a subject in need thereof for inhibition of proliferation of retinal pigment epithelium or a disease associated therewith. The related U.S. Pat. No. 6,075,032 is directed to inhibition of choroidal neovascularization by contacting RPE cells with an AP-1 antagonist. The related U.S. Pat. No. 5,824,685 regards amelioration of proliferative vitreoretinopathy or traction retinal detachment by contacting RPE cells with a retinoic acid receptor selected from ethyl-6-[2-(4,4-dimethylthiochroman-6-yl)ethynyl]nicotinate, 6-[2-(4,4-dimethylchroman-6-yl)ethynyl]nicotinic acid, and p-[(E)-2-(5,6,7,8-tetrahydro-5,5,8,8-tetramethyl-2-naphthyl)propenyl]-benzoic acid. The related U.S. Pat. No. 6,372,753 addresses inhibition of an ocular disease resulting from proliferation of retinal pigment epithelium by providing at least one AP-1 antagonist and at least one retinoic acid receptor (RAR) agonist, except for retinoic acid.

WO 01/58494 is directed to treating or preventing an ocular disease, such as age-related macular degeneration, by contacting an ocular cell with an expression vector comprising a nucleic acid sequence encoding an inhibitor of angiogenesis and a neurotrophic agent. In specific embodiments, the inhibitor of angiogenesis and the neurotrophic agent are one and the same, such as pigment epithelium-derived factor (PEDF).

WO 02/13812 regards the use of an insulin-sensitizing agent, preferably peroxisome proliferator-activated receptor-γ (PPAR γ) agonists, for the treatment of an inflammatory disease, such as an ophthalmic disease.

WO 00/52479 addresses diagnosing, treating, and preventing drusen-associated disorders (any disorder which involves drusen formation), including AMD. In specific embodiments, there are methods related to providing an effective amount of an agent that inhibits immune cell proliferation or differentiation, such as antagonists of TNF-alpha.

Thus, the present invention provides a novel approach to reduce lipid content of ocular tissue, such as Bruch's membrane and further provides methods and compositions for the treatment of macular degeneration, such as AMD.

### BRIEF SUMMARY OF THE INVENTION

The present invention is directed to a system, method, and/or composition(s) related to treating AMD. Treatments for dry AMD have been lacking, because the pathogenesis of this common condition is poorly understood, and the inventors have demonstrated analogous biological behavior between human retinal pigment epithelial (RPE) cells and macrophages that point toward similar pathogenic mechanisms of AMD and atherosclerosis. Specifically, reverse cholesterol transport (RCT) is exploited in the present invention for the treatment of AMD. The present inventors provide the novel demonstration of RCT in RPE cells in the eye. More specifically, RCT is regulated through manipulation of levels of cholesterol and/or phospholipid transporters (ABCA-1, Apo E, SRB-1, SRB-2) by nuclear hormone receptor ligands such as agonists of thyroid hormone (TR), liver X receptor (LXR), and/or retinoid X receptor (RXR). A goal for the present invention is the reduction of lipid content of RPE Bruch's membrane to facilitate an improvement in visual function and/or, in some embodiments, prevent ocular disease, such as AMD. Reduction of the lipid content of Bruch's membrane preferably results in at least one or more of the following: reduction in development of CNV; improvement in dark adaptation; improvement in night vision; improved visual acuity; and/or improved recovery to bright flash stimulus.

In a specific embodiment of the present invention, patients with drusen and no evidence of choroidal neovascularization are administered a nuclear hormone agonist, such as thyroid hormone (TR) agonist (for example, T3 (3,5,3′-L-triiodothyronine), TRIAC (3-triiodothyoacetic acid), GC1, KB-000,141 and/or KB141 (Karo Bio; Huddinge, Sweden). Administration could be orally or by sustained release systems well known in the art. In a specific embodiment, the agonist binds to at least one nuclear hormone receptor in the RPE and induces upregulation of RCT. Efflux of lipid from RPE increases, and the likelihood of visual loss from choroidal neovascularization is reduced. Other nuclear hormone receptor ligands of the TR, RXR, and LXR families, for example, which are well known in the art, are used independently or in combination with each other to enhance RCT by RPE.

In an embodiment of the present invention, there is a method of increasing lipid efflux from an ocular tissue, comprising the step of delivering to the tissue a nuclear hormone receptor ligand. In a specific embodiment, ocular tissue is retinal pigment epithelium (RPE), Bruch's membrane, or a combination thereof. In another specific embodiment, the nuclear hormone receptor is thyroid hormone receptor. In an additional specific embodiment, the ligand of thyroid hormone receptor is 3,5,3′-L-triiodothyronine (T3), TRIAC (3-triiodothyroacetic acid); KB141; GC-1; 3, 5 dimethyl-3-isopropylthyronine; or a mixture thereof. In one embodiment, the nuclear hormone receptor is liver X receptor. In a specific embodiment, the ligand of liver X receptor is 22 (R) hydroxycholesterol; acetyl-podocarpic dimer; T0901317; GW3965 (12); 24(S),25-epoxycholesterol; 24(R),25-epoxycholesterol; 22(R)-ol-24(S),25-epoxycholesterol; 22(S)-ol,24(R),25-epoxycholesterol; 24(S),25-iminocholesterol; methyl-H-cholenate; dimethyl-hydroxycholenamide; 24(S)-hydroxycholesterol; 24(R)-hydroxycholesterol; 22(S)-hydroxycholesterol; 22(R),24(S)-dihydroxycholesterol; 25-hydroxycholesterol; 24(S),25-dihydroxycholesterol; 24(R),25-dihydroxycholesterol; 24,25-dehydrocholesterol; 7(a)-ol,24(S),25-epoxycholesterol; 7(b)-ol,24(S),25-epoxycholesterol; 7k,24(S),25-epoxycholesterol; 7(α)-hydroxycholesterol; 7-ketocholesterol; cholesterol; 5,6-24(S),25-diepoxycholesterol; or a mixture thereof. In a specific embodiment, the nuclear hormone receptor is retinoid X receptor, ligands of which include 9 cis-retinoic acid; AGN 191659 [(E)-5-[2-(5,6,7,8-tetrahydro-3,5,5,8,8-pentamethyl-2-naphthyl)propen-1-yl]-2-thiophenecarboxylic acid]; AGN 191701 [(E) 2-[2-(5,6,7,8-tetrahydro-3,5,5,8,8-pentamethyl-2-naphthyl)propen-1-yl]-4-thiophene-carboxylic acid]; AGN 192849 [(3,5,5,8,8,-pentamethyl-5,6,7,8-tetrahydronaphthalen-2-yl) (5 carboxypyrid-2-yl)sulfide]; LGD346; LG100268; LG100754; BMS649; bexaroteneR (4-[1-(5,6,7,8-tetrahydro-3,5,5,8,8-pentamethyl-2-naphthalenyl) ethenyl] benzoic acid); or a mixture thereof. In one embodiment, the ocular tissue is comprised in an individual, such as one at risk for developing macular degeneration or another ocular disease, and/or the ocular tissue is comprised in individuals afflicted with macular degeneration (for example, age-related macular degeneration). In other specific embodiment, the individual is afflicted with Stargardts disease (fundus flavimaculatus) or is at risk for developing Stargardts disease.

In another embodiment of the present invention, there is a method of increasing reverse cholesterol transport in an ocular tissue, comprising the step of delivering to the tissue at least one ligand of a nuclear hormone receptor. In a specific embodiment, the ocular tissue is retinal pigment epithelium (RPE), Bruch's membrane, or a combination thereof.

In an additional embodiment of the present invention, there is a method of treating macular degeneration (AMD) in an individual, comprising the step of delivering to the individual a ligand of a nuclear hormone receptor. In a specific embodiment, the delivering occurs under conditions wherein reverse cholesterol transport is upregulated.

In another embodiment of the present invention, there is a kit for the treatment of macular degeneration, housed in a suitable container, comprising a ligand of a nuclear hormone receptor. In a specific embodiment, the nuclear hormone receptor is TR, RXR, LXR, or a combination thereof. In some embodiments, it is useful to comprise a combination of nuclear hormone receptors, since it is well known in the art that many are heterodimers (for example, LXR and RXR, or RXR and TR). In a specific embodiment, the kit comprises a pharmaceutically acceptable excipient. In another specific embodiment, the ligand for a nuclear hormone receptor is comprised in the pharmaceutically acceptable excipient.

In an additional embodiment of the present invention, there is a method of treating macular degeneration (AMD) in an individual, comprising the steps of identifying a ligand for a nuclear hormone receptor; and delivering said ligand to said individual. Methods to identify a ligand for a nuclear hormone receptor are well known in the art.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows that RPE cells express Apo E, ABCA1, and LXR α.

FIG. 2 shows RPE cell expression of SR-BI and SR-BII.

FIG. 3 illustrates SR-BI and SR-BII immunofluorescence in RPE cells.

FIG. 4 demonstrates ABCA1 immunofluorescence in RPE cells.

FIG. 5 demonstrates that basal Apo E expression is greater than apical Apo E expression in cultured human RPE cells.

FIG. 6 shows regulation of Apo E expression by nuclear hormone receptor ligands.

FIG. 7 provides a non-denatured polyacrylamide gel of lipoprotein fractions.

FIG. 8 shows ¹⁴C distributeion of the fractions from FIG. 7.

FIG. 9 demonstrates thin layer chromatography illustrating the identification of six out of seventeen spots of an HDL fraction. Note: HDL is the high density lipoprotein fraction; POS is labeled POS starting material; PC is phophatidylcholine; PI is phosphatidylinisotol; PE is phosphatidylethanolamine; C is cholesterol; TRL is TG rich lipid, including triglycerides and cholesterol ester.

FIG. 10 demonstrates that ¹⁴C counts increase following drug treatments that increase RCT.

FIG. 11 illustrates ABCA1 regulation by RXR and LXR ligands.

FIG. 12 shows HDL, LDL and plasma stimulation of ¹⁴C-labeled lipid transport the identification of HDL from RPE cells.

FIG. 13 shows stimulation of CD36 expression by oxidized lipid.

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “age-related macular degeneration” as used herein refers to macular degeneration in an individual over the age of about 50. In one specific embodiment, it is associated with destruction and loss of the photoreceptors in the macula region of the retina resulting in decreased central vision and, in advanced cases, legal blindness.

The term “Bruch's membrane” as used herein refers to a five-layered structure separating the choriocapillaris from the RPE.

The term “increase lipid efflux” or “increasing lipid efflux” as used herein refers to an increased level and/or rate of lipid efflux, promoting lipid efflux, enhancing lipid efflux, facilitating lipid efflux, upregulating lipid efflux, improving lipid efflux, and/or augmenting lipid efflux. In a specific embodiment, the efflux comprises efflux of phospholipid, triglyceride, cholesterol, and/or cholesterol ester.

The term “macula” as used herein refers to the light-sensing cells of the central region of the retina.

The term “macular degeneration” as used herein refers to deterioration of the central portion of the retina, the macula.

The term “nuclear hormone receptor” as used herein refers to an intracellular receptor that plays a role in expression of gene(s) involved in physiological processes, examples of which include cell growth and differentiation, development, and homeostasis. In a specific embodiment, these receptors are members of a superfamily of receptors, whose members recognize similar DNA sequences that contain two or more hexanucleotide DNA-binding half-sites arranged as direct repeats or inverted repeats. It is through this recognition that these receptors are able to regulate the expression of genes in the nucleus, and thereby regulate the respective physiological process. Examples of nuclear hormone receptors include thyroid hormone receptor, liver X receptor, retinoid X receptor, estrogen receptor, androgen receptor, peroxisome proliferator activated receptors (PPARs), trans-retinoic acid receptor (RAR), the vitamin D receptor (VDR), glucocorticoid receptor, the progesterone receptor, and isoforms thereof.

Nuclear hormone receptors in some embodiments include those that remain sequestered in the cytoplasm in the absence of their cognate ligands (e.g., steroid hormone receptors). Upon binding of the ligand, the steroid hormone receptors are translocated to the nucleus where they bind to hormone response elements, typically as homodimers.

In other embodiments the nuclear hormone receptors are not sequestered in the cytoplasm in the absence of their ligands, but rather remain in the nucleus. These receptors, which include the thyroid hormone, retinoid, fatty acid, and eicosanoid receptors, typically bind to their cognate response elements as heterodimers with, for example, a 9-cis-retinoic acid receptor (RXR). Often, binding of a nuclear receptor to a response element occurs in the absence of the cognate ligand. An example of such a nuclear receptor is the famesoid X receptor (FXR).

Methods to identify ligands of nuclear hormone receptors are well known in the art, examples of which are described in U.S. Pat. No. 5,846,711 and U.S. Pat. No. 6,266,622, both of which are incorporated by reference herein in their entirety.

The term “reverse cholesterol transport” as used herein refers to transport of cholesterol from peripheral tissues to the liver. In a specific embodiment, it refers to efflux of lipid from RPE cells. In specific embodiments, it comprises efflux of cellular cholesterol and/or phospholipid to HDL, and, in further specific embodiments, it comprises HDL delivery of cholesterol ester to the liver, such as for biliary secretion.

The term “upregulate” as used herein is defined as increasing the level and/or rate of an event, process, or mechanism, such as reverse cholesterol transport.

II. The Present Invention

As stated, the histopathology of macula in patients with AMD shows diffuse thickening of Bruch's membrane, and the overlying RPE is attenuated and full of lipofuscin granules. Photoreceptors are shortened and atrophic, and much of the thickened Bruch's membrane consists of lipid deposition. It is known that following about 50 years of age, the rate of lipid accumulation accelerates (Holz et al., 1994).

Using cell culture methods to study lipid metabolism, the inventors have, shown a number of analogous mechanisms for lipid metabolism that are shared by macrophages and human RPE cells. The shared biology of these two cell types indicates useful therapeutic approaches for treatment of AMD. Specifically, the present inventors are the first to show that RCT occurs in RPE cells, and enhancement of RCT is beneficial for removing undesired lipid from the RPE cells and/or Bruch's membrane to facilitate retinal metabolism. In a specific embodiment, the transporters in the RCT system are regulated to improve RCT. In a further specific embodiment, this regulatory aspect of the present invention provides a novel treatment for AMD.

Although there has been discussion in the field regarding mechanisms of lipid accumulation in macula of AMD individuals, the present invention regards efflux of lipid into the circulation, which reduces the amount of lipid in RPE and/or Bruch's membrane. Promotion of this efflux comprises one aspect of the invention and is an effective therapy for both early and late AMD. A skilled artisan recgonizes that early AMD comprises the presence of drusen and late stage AMD comprises visual loss from choroidal neovascularization or geographic atrophy.

Thus, the present invention provides the novel idea in the field in which reverse cholesterol transport occurs in RPE cells. In specific embodiments, the invention provides methods and compositions related to facilitating efflux of cholesterol and/or phospholipids from inside an RPE cell to the outside of the RPE cell, and further through Bruch's membrane. In another specific embodiment, following efflux from Bruch's membrane the cholesterol and/or phospholipids are transported by apolipoprotein E, apolipoprotein A1, and other transporters, or a combination thereof, to HDL for removal to the liver.

A skilled artisan recognizes the important role reverse cholesterol transport (RCT) plays in lipid homeostasis. HDL levels are inversely correlated with incidence of coronary artery disease (CAD). Tangier's disease, which comprises a mutation of ABCA1, leads to deposition of cholesterol in reticuloendothelial tissues and premature atherosclerosis. Furthermore, the Apo E null mouse is an excellent model of atherosclerosis and hyperlipidemia. Interestingly, supporting an important role of Apo E in RCT, reconstitution of Apo E positive macrophages via bone marrow transplant into an Apo E null mouse prevents atherosclerosis. This occurs in spite of persistent hyperlipidemia.

In one embodiment of the present invention a transporter of lipid from RPE cells is enhanced for the transport activity, such as by an increase in the level of the transporter. Examples of transporters include apo E, ABCA1, SR-BI, SR-BII, ABCA4, ABCG5, ABCG8; other proteins that might be involved are LCAT, CETP, PLTP, LRP receptor, LDL receptor, Lox-1, and lipases. In a specific embodiment, lox-1 and PLTP are expressed in RPE, as demonstrated by RT_PCR. In a specific embodiment of the present invention, apo A1 is utilized to facilitate RCT from RPE cells. In an additional specific embodiment, apo A1 is made by RPE cells.

In a specific embodiment of the present invention, strategies for intervention for treatment of AMD are provided in which reverse cholesterol transport is enhanced at the level of the RPE by upregulating ABCA1, Apo E, SR-BI and/or SR-BII expression. SR-B has been reported to be upregulated by 17beta-Estradiol and testosterone. Additionally, or alone, HDL binding to effluxed lipids is enhanced, thereby increasing efflux of lipids from Bruch's membrane into the circulation and providing therapy for AMD. In one embodiment, an increase in HDL levels is utilized to facilitate lipid efflux from RPE cells and/or Bruch's membrane, and in a specific embodiment, levels of specific subspecies of HDL are utilized to facilitate lipid efflux. For example, effluxed lipids could bind to preβ-HDL, HDL1, HDL2 or HDL3. Effluxed lipids could also bind prebeta-1, prebeta-2, prebeta-3, and/or prebeta-4 HDL. In a specific embodiment, the effluxed lipids bind preferentially to HDL2 that comprises apo E.

One skilled in the art recognizes particular RCT components are present in RPE cells (Mullins et al., 2000; Anderson et al., 2001). Nuclear hormone receptors known to regulate expression of reverse cholesterol transport proteins are also expressed in cultured human RPE. Thus, in a preferred embodiment of the present invention, ligands to at least one of the nuclear hormone receptors upregulates RCT. In further embodiments, following efflux from RPE cells, the lipids bind HDL, so in an embodiment of the present invention there is upregulation of HDL for AMD treatment, such as by statins and/or niacin.

In an alternative embodiment, treatment for AMD comprises reduction of RCT. For example, in individuals past a certain age, such as about 50, 55, 60, 65, 70, 75, 80, and so on, the transporters are preferentially inhibited. In one aspect of this embodiment, HDL is unable to enter Bruch's membrane to remove the lipids and the RPE continues to efflux lipids. In such cases where effluxed lipids from RPE cannot be removed by a lipoprotein acceptor, lipid efflux by RPE is inhibited to maintain macromolecular transport across Bruch's membrane. Inhibition of RCT by reducing levels of ABCA-1, apo E, and/or SRB-1, or SRB-2 would reduce accumulation of lipid in Bruch's membrane.

In embodiments of the present invention, ligands for nuclear hormone receptors are utilized as compounds for enhancing RCT for the reduction of lipid content of RPE and Bruch's membrane. In a specific embodiment, the nuclear hormone receptor ligands are utilized for treatment of AMD. In a further specific embodiment, the nuclear hormone receptors comprise TR, RXR, and/or LXR. In other specific embodiments, ligands of the nuclear hormone receptors are delivered to at least one RPE cell to facilitate efflux of lipids from the RPE cell and/or are delivered to Bruch's membrane for efflux from Bruch's membrane. Examples of ligands for TR include T3 (3,5,3′-L-triiodothyronine). Other examples of TR ligands include but are not limited to TRIAC (3-triiodothyroacetic acid); KB141 (Karo Bio); GC-1; and 3, 5 dimethyl-3-isopropylthyronine. Examples of ligands for RXR include 9 cis-retinoic acid, and other RXR ligands also include but are not limited to: AGN 191659 [(E)-5-[2-(5,6,7,8-tetrahydro-3,5,5,8,8-pentamethyl-2-naphthyl)propen-1-yl]-2-thiophenecarboxylic acid]; AGN 191701 [(E) 2-[2-(5,6,7,8-tetrahydro-3,5,5,8,8-pentamethyl-2-naphthyl)propen-1-yl]-4-thiophene-carboxylic acid]; AGN 192849 [(3,5,5,8,8,-pentamethyl-5,6,7,8-tetrahydronaphthalen-2-yl) (5 carboxypyrid-2-yl)sulfide]; LGD346; LG100268; LG100754; BMS649; and bexaroteneR (Ligand Pharmaceuticals) (4-[1-(5,6,7,8-tetrahydro-3,5,5,8,8-pentamethyl-2-naphthalenyl) ethenyl] benzoic acid). Examples of ligands for LXR include 22 (R) hydroxycholesterol, acetyl-podocarpic dimer, T0901317, and GW3965.

In an embodiment of the present invention, expression of a sequence is monitored following administration of an upregulator of its expression or a compound suspected to be an upregulator. A skilled artisan recognizes how to obtain these sequences, such as commercially from Celera Genomics, Inc. (Rockville, Md.) or from the National Center for Biotechnology Information's GenBank database. Exemplary apo E polynucleotide sequences include the following, cited with their GenBank Accession number: SEQ ID NO:1 (K00396); SEQ ID NO:2 (M10065); and SEQ ID NO:3 (M12529). Some exemplary apo E polypeptide sequences include the following, cited with their GenBank Accession number: SEQ ID NO:4 (AAB59546); SEQ ID NO:5 (AAB59397); and SEQ ID NO:6 (AAB59518).

In other embodiments, sequences of ABCA-1 are utilized, such as to monitor ABCA-1 expression related to methods of the present invention. Some examples of ABCA1 polynucleotides include SEQ ID NO:7 (NM_{—}005502); and SEQ ID NO:8 (AB055982). Some examples of ABCA1 polypeptides include SEQ ID NO:9 (NP_{—}005493); and SEQ ID NO:10 (BAB63210).

In some methods of the present invention, expression levels of sequences of SR-BI and SR-B2 polynucleotides are monitored following administration of a nuclear hormone receptor ligand. An example of SR-BI polynucleotide is SEQ ID NO:11 (NM_{—}005505) and an example of a SR-BI polypeptide is SEQ ID NO:12 (NP_{—}005496).

III. Pharmaceutical Compositions and Routes of Administration

Compositions of the present invention may have an effective amount of a compound for therapeutic administration and, in some embodiments, in combination with an effective amount of a second compound that is also an anti-AMD agent. In a specific embodiment, the compound is a ligand/agonist of a nuclear hormone receptor. In other embodiments, compounds that upregulate expression of HDL are the compounds for therapeutic administration. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-AMD agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The delivery vehicles of the present invention may include classic pharmaceutical preparations. Administration of these compositions according to the present invention will be via any common route so long as the target ocular tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions. In some embodiments, the compositions are administered by sustained release intra- or extra-ocular devices.

The vehicles and therapeutic compounds therein of the present invention are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for AMD treatment, diagnosis and/or prevention may be assembled together in a kit. When the components of the kit are provided in one or more liquid solutions, the liquid solution preferably is an aqueous solution, with a sterile aqueous solution being particularly preferred.

For in vivo use, an anti-AMD agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the anti-AMD composition.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in eye drops, cremes and lotions.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intraocular, intravenous, intramuscular, and subcutaneous administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of ocular tissues may be accomplished in any one of a variety of ways. In one embodiment, there is the use of liposomes to target a compound of the present invention to the eye, and preferably to RPE cells and/or Bruch's membrane. For example, the compound may be complexed with liposomes in the manner described above, and this compound/liposome complex injected into patients with AMD, using intravenous injection to direct the compound to the desired ocular tissue or cell. Directly injecting the liposome complex into the proximity of the RPE or Bruch's membrane can also provide for targeting of the complex with some forms of AMD. In a specific embodiment, the compound is administered via intra-ocular sustained delivery (such as Vitrasert® or Envision® by Bauch and). In a specific embodiment, the compound is delivered by posterior subtenons injection. In another specific embodiment, microemulsion particles with apo E (such as, recombinant) are delivered to ocular tissue to take up lipid from Bruch's membrane, RPE cells, or both.

Those of skill in the art will recognize that the best treatment regimens for using compounds of the present invention to treat AMD can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient. Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

IV. Kits

Any of the compositions described herein may be comprised in a kit. In a non-limiting example, a nuclear hormone receptor agonist or ligand, and in some embodiments, at least one additional agent, may be comprised in a kit.

The kits may comprise a suitably aliquoted nuclear hormone receptor ligand, and/or additional agent compositions of the present invention, whether labeled or unlabeled, as may be used to prepare a standard curve for treatment of macular degeneration, such as AMD. The components of the kits may be packaged in aqueous media or in lyophilized form. When reagents and/or components are provided as a dry powder, the powder can be reconstituted by the addition of a suitable solvent. It is envisioned that the solvent may also be provided in another container means.

The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the nuclear hormone receptor ligand, additional agent, and any other reagent containers in close confinement for commercial sale. Such containers may include injection or blow-molded plastic containers into which the desired vials are retained.

### EXAMPLES

The following is an illustration of preferred embodiments for practicing the present invention. However, they are not limiting examples. Other examples and methods are possible in practicing the present invention.

### Example 1

### Materials and Methods

Cell Culture and Drug Treatments

Primary cultures of normal human RPE cells from passages 5 to 10 were used for the experiments described. RPE cells were grown to confluence on laminin-coated 6 well Transwell tissue culture plates (Costar) with DMEM-H21 containing 10% fetal bovine serum, 2 mM glutamine, 50 μg/ml gentamicin and 2.5 mg/ml fungizone in the top and bottom chambers. For immunofluorescent staining cells were grown on laminin coated slides in the same medium. Cells were grown for at least 1 week at confluence prior to drug treatment. Cells to be treated with drugs were incubated in serum free DMEM-H21 prior to drug addition. Drug treatments were in serum free DMEM-H21 with or without 10⁻⁷ M thyroid hormone (T₃), 2.5×10⁻⁶ M 22 (R) hydroxycholesterol, or 10⁻⁷ M cis retinoic acid in both chambers for 36 hours.

RT-PCR

Confluent cell cultures were harvested and total RNA was purified using RNAzol (Teltest, Inc., Friendswood, Tex.) according to the manufacturer's instructions. Equal amounts of purified RNA were used in each reaction as templates for cDNA synthesis using the 1st Strand Synthesis Kit for RT-PCR (AMV) (Boehringer, Indianapolis, Ind.). RT-PCR was carried out on 1 μg of cDNA with Amplitaq Taq polymerase (Perkin-Elmer, Branchburg, N.J.). In some experiments apo E RT-PCR products were quantified using the QuantumRNA assay kit according to the manufacturer's instructions (Ambion, Austin, Tex.). Briefly, 18S rRNA and apo E cDNAs are simultaneously amplified in each reaction. The RT-PCR products are resolved by electrophoresis on 1.4% agarose gels. The apo E mRNA expression is assessed relative to the internal 18S rRNA expression by densitometric analysis of photographed agarose gels.

RT-PCR primers specific to human apo E, ABCA1, SR-BI, SR-BII, and 1xr a were used. The RT-PCR product of the predicted sizes for the apo E, ABCA1, SR-BI, and SR-BII RT-PCR products were excised form the gel and their identities were confirmed by DNA sequencing (not shown).

Immunofluoresence Microscopy

RPE cells, grown on slides, were σταινεδ with either antisera to ABCA1, or with purified antibodies to SR-BI or SR-BII. Cells were fixed in ice cold 100% MeOH for 20 min. All subsequent steps were performed at room temperature. Cells were washed in phosphate buffered saline (PBS) and incubated for in 5% goat serum in PBS for 30 min. Cells were then washed in buffer A (150 mM NaCl, 10 mM phosphate, pH 7.8) and incubated with the primary antibody in buffer A for 45 min. After washing with buffer A the cells were incubated in Avidin Blocking Reagent (Vector Laboratories, Burlingame, Calif.) for 15 min, washed in buffer A again and incubated in Biotin Blocking Reagent (Vector Laboratories, Burlingame, Calif.) for 15 min. After washing in buffer A, cells were incubated in 10 μg/ml biotinylated goat anti-rabbit IgG (Vector Laboratories, Burlingame, Calif.) in buffer A for 30 min, washed in buffer A and incubated in 20 μg/ml fluorescein conjugated avidin D (Vector Laboratories, Burlingame, Calif.) in buffer B (150 mM NaCl, 100 mM sodium bicarbonate, pH 8.5) for 30 min. The cells were washed in buffer B and a cover slip was added to each slide, over a few drops of Vectashield (Vector Laboratories, Burlingame, Calif.). The slides were stored in the dark until ready for microscopic examination.

Apo E Western Blotting

Cells were treated with Media was concentrated 20-fold by centrifugal ultrafiltration (VIVA SPIN 20, MCO 5,000, Viva Sciences, Hannover, Germany), dialyzed against 0.15M NaCl, 1 mM sodium EDTA, 0.025% sodium azide (SalEN). Total protein content was determined by a modified Lowry assay (BioRad DC kit, Richmond, Calif.). Concentrated media (50 μg protein) was made to Start Buffer (0.025 M NaCl, 0.010 M tris (pH 8.5), 5 mM MnCl₂) and adsorbed onto a 0.1 ml column containing Heparin-Sepharose CL-4B (Pharmacia, Uppsala, Sweden). Following a 2 ml wash in Start Buffer, the apo E containing bound fraction was eluted with 0.5M NaCl in Start buffer. The eluate was concentrated to 20 μl and buffer-exchanged to SalEN by centrifugal ultrafiltration (Biomax, 5k MCO, Millipore, Bedford, Mass.). Apo E was resolved by tris-tricine buffered SDS-PAGE (5-25% linear acrylamide gradient) and proteins electrophoetically transferred (55V, 18 h) to nitrocelluose membrane filters (Schleicher and Shuell, Keen, N H). Membranes were blocked with 10% bovine serum albumin at room temperature and probed with 1% goat anti-human apo E antiserum (18 h, 3° C.) prepared in 0.15% NaCl, 1 mM EDTA (pH 7.4), 0.1% Triton X-100 (SalET). The primary-bound anti-apo E antibodies were detected calorimetrically with horseradish peroxidase conjugated rabbit anti-goat Ig (H+L) and NiCl₂-enhanced diaminobenzine staining. Stained bands were compared densitometrically from the digitized scanned image (NIH Image, v.1.62). Anti apo E antibodies were obtained by hyper-immunization of goats with purified apo E or obtained from Assay Designs (A299, Ann Arbor, Mich.)

Lipoprotein fractions were prepared from conditioned media that was adjusted with solid KBr to a density of 1.21 g/ml. Samples were ultracentrifuged in a Beckman 42.2 Ti rotor at 40,000 rpm for 18 h at 110° C. The lipoprotein and lipoprotein-free fractions, the top and bottom 50 μl, respectively, were dialysed against SalEN prior to analysis.

[¹⁴C] Docosohexanoic Acid (DHA) Labeled POS Uptake and Transport

Bovine outer photoreceptor outer segments (POS) were labeled by incubating for with Coenzyme A, ATP, Mg²⁺, and [¹⁴C]-DHA. Cells grown on laminin coated Transwell plates were incubated with 12 μg/ml labeled POS in the apical chamber for 36 hours in medium containing 10% lipoprotein deficient fetal bovine serum. The basal medium was subjected to scintillation counting to determine the amount of [¹⁴C] labeled lipids transported through the RPE cells.

Identification of Acceptors for Exported ¹⁴C Lipids

Bovine outer photoreceptor outer segments (POS) were labeled by incubating for with Coenzyme A, ATP, Mg²⁺, and [¹⁴C]-DHA. Cells grown on laminin coated Transwell plates were incubated with 12 μg/ml labeled POS in the apical chamber for 36 hours in medium containing 10% lipoprotein deficient fetal bovine serum. The basal chambers contained either 1 mg/ml human HDL, 1 mg/ml human LDL or 100% human plasma. The basal medium was collected and lipoproteins were repurified from by potassium bromide density gradient centrifugation at d=1.21 g/ml (Beckman 42.2 Ti rotor, 40,000 rpm, 18 h, 10° C.), dialyzed, and resolved by size in nondenaturing 0-35% PAGE. Gels were stained with coomassie blue R-250. Gel lanes were sectioned into thirty 2 mm slices that were digested (TS-1, Research Products International) and radioactivity quanitfied by liquid scintillation spectrometry.

### Example 2

### Expression of Transporters in RPE Cells

One skilled in the art recognizes that certain RCT components in cultured human RPE cells have been demonstrated (Mullins et al., 2000; Anderson et al., 2001). Nuclear hormone receptors known to regulate expression of reverse cholesterol transport proteins are also expressed in cultured human RPE.

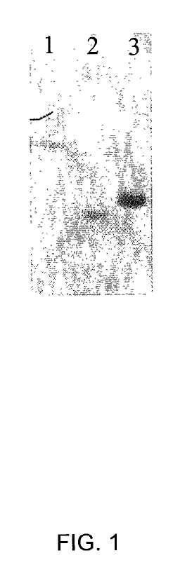

A skilled artisan recognizes that there is expression of TRs and RXRs in RPE cells in culture (Duncan et al. 1999). RT-PCR of human RPE cell cDNA revealed that these cells also express mRNAs for apo E, ABCA1, SR-BI, SR-BII and 1xr α. As shown in FIG. 1 lane 1, FIG. 1 lane 2 and FIG. 1 lane 3, RPE cells express mRNAs for apo E, ABCA1 and 1xr α, respectively.

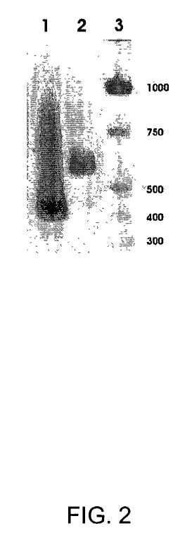

As shown in FIG. 2, lane 1, and FIG. 2, lane 2, RPE cells express mRNA for SR-BI and SR-BII respectively.

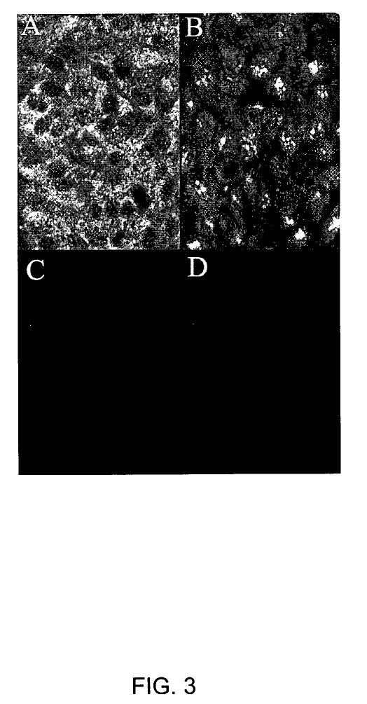

Furthermore, in immunofluoresence microscopy experiments, RPE cells stain strongly for SR-BI (FIG. 3A) and SR-BII (FIG. 3B). Control non-specific IgG or antibody vehicle did not stain RPE cells (FIGS. 3C and 3D, respectively). Expression of SR-BI and SR-BII in these cells was confirmed by PCR.

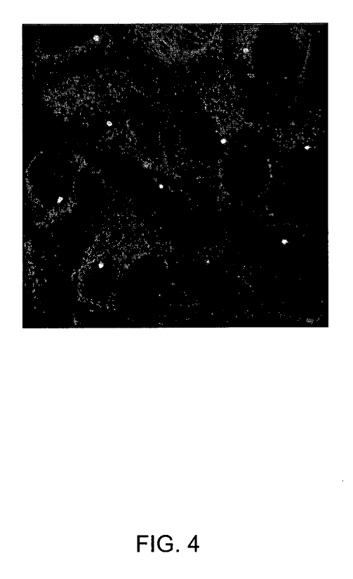

Expression of ABCA1 protein was demonstrated by immunofluorescent staining of RPE cells with an antibody to ABCA1 (FIG. 4). Cell nuclei were stained with DAPI.

### Example 3

### Regulation of Apo E Secretion in RPE Cells

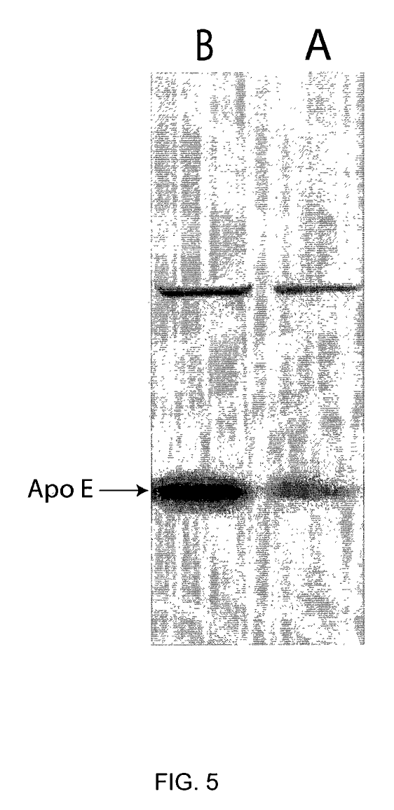

In order to distinguish apical (A) from basally (B) secreted apo E, RPE cells were cultured on laminin-coated Transwell plates. Specifically, human cultured RPE (passage 2-10, 35 y.o. donor) were placed on laminin-coated Transwell plates, wherein the upper and lower wells both had serum-free media. Total protein and apo E-specific protein concentrations were measured from media pooled and concentrated from 3-6 replicate wells. To assess apo E-specific secretion, apo E was purified from conditioned media by heparin-sepharose affinity chromatography and visualized by western blotting. Apo E concentrations were consistently greater in the basolateral media (FIG. 5, lane 1 vs. lane 2). These data demonstrate that RPE cells display polarized secretion of cellular proteins, including apo E. Thus, this indicated that Apo E is preferentially secreted basally, supporting its role in RCT.

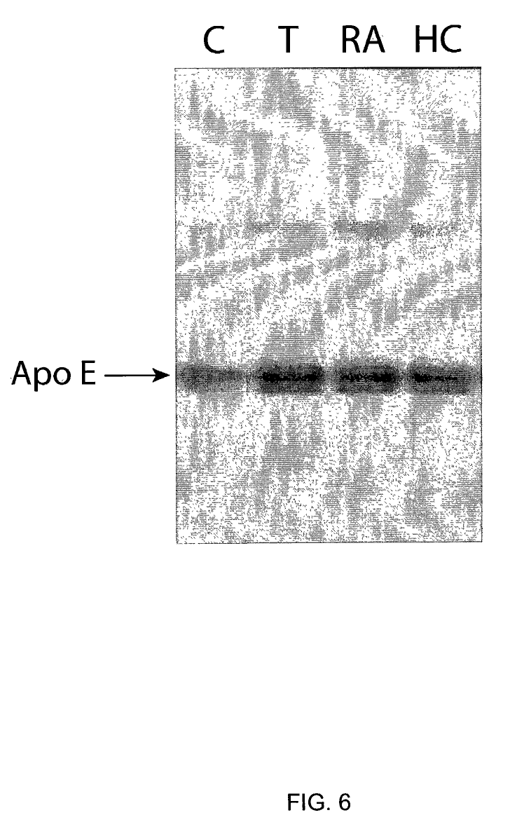

Since RPE cells express 1xr α as well as thyroid hormone receptors (TRs) and retinoid-X-receptors (RXRs), the effect of 10⁻⁷ M T3, 2.5×10⁻⁶ M 22 (R) hydroxycholesterol (HC) (an 1xr α agonist), or 10⁻⁷ M cis retinoic acid (cRA) (an RXR agonist) on apo E secretion from RPE cells was tested. FIG. 6 illustrates the same experimental procedure as described above, but with basal and apical media both containing the following compounds for a 36 hour incubation: T3 (10⁻⁷) M (T); 9 cis-RA (10⁻⁶) M (RA); and 22 (R) hydroxycholesterol 2.5 (10⁻⁶) M (HC). The basal media was analyzed for Apo E expression with Western blot, and the results showed increased basal expression of Apo E with the compound treatments. Thus, as before, polarized apo E secretion was observed and, in this case, occurred in the presence of T3, HC or cRA, indicating that an increase in levels of basally secreted apo E is the result of administration of these compounds to RPE cells.

### Example 4

### Assay of Efflux from RPE Cells

This example characterizes efflux of POS residues from RPE cells, particularly regarding binding to HDL. Giusto et al. (1986) describes a method of ¹⁴C decoshexanoic acid (DHA) labeling of bovine photoreceptor outer segment (POS) lipids. Generally, an approximately 36 hour incubation over human RPE cells wherein the basal medium contains plasma, HDL, or LDL is followed by centrifugation of the basal media to collect lipoprotein fraction, which is then analyzed to determine distribution of radioactivity.

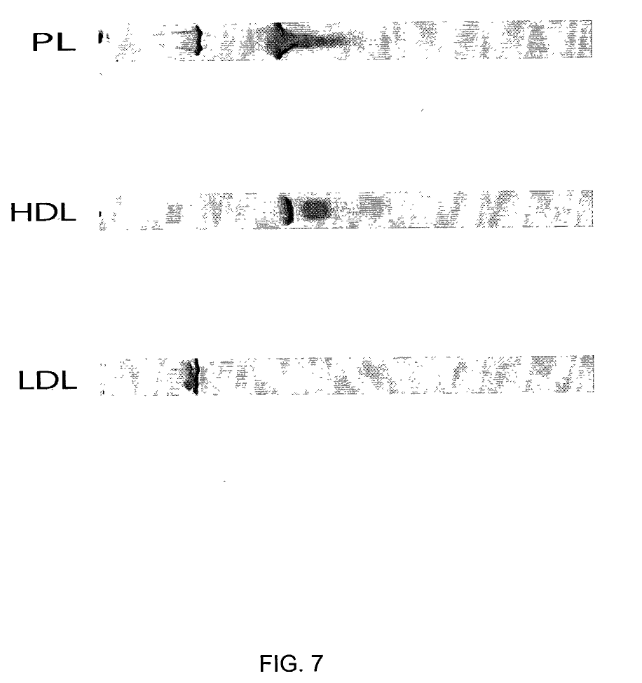

Specifically, bovine photoreceptor outer segment (POS) are labeled with ¹⁴C decoshexanoic acid (DHA) and placed in lipoprotein deficient media. Following this, they are placed over cultured human RPE on Transwell plates for 36 hours, and the basal medium contained either 100% plasma, HDL (1 mg/cc) or LDL (1 mg/cc). After 36 hours, basal media was centrifuged to collect lipoprotein fraction (density 1.2). This fraction was then run on a non-denaturing gel and stained with Coomassie blue. FIG. 7 shows LDL and HDL fractions, both separately and together in plasma (PL). The PL fraction contains the same amount of HDL and LDL as each of the separated fractions (HDL, LDL).

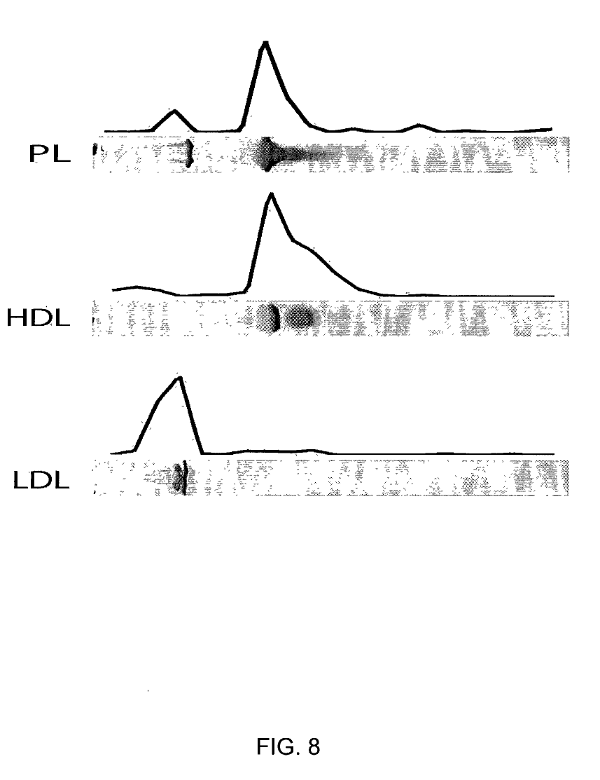

The PA gel was cut into about 1 mm pieces, and the radioactivity distribution was determined (FIG. 8). With either LDL or HDL alone, counts were observed over respective lipoprotein fractions. When both LDL and HDL in plasma are present, counts localize preferentially over HDL fraction. This indicates that following phagocytosis of POS by RPE, POS residues are effluxed and preferentially bound by HDL. This is a novel demonstration illustrating that RCT to an HDL acceptor occurs in RPE cells.

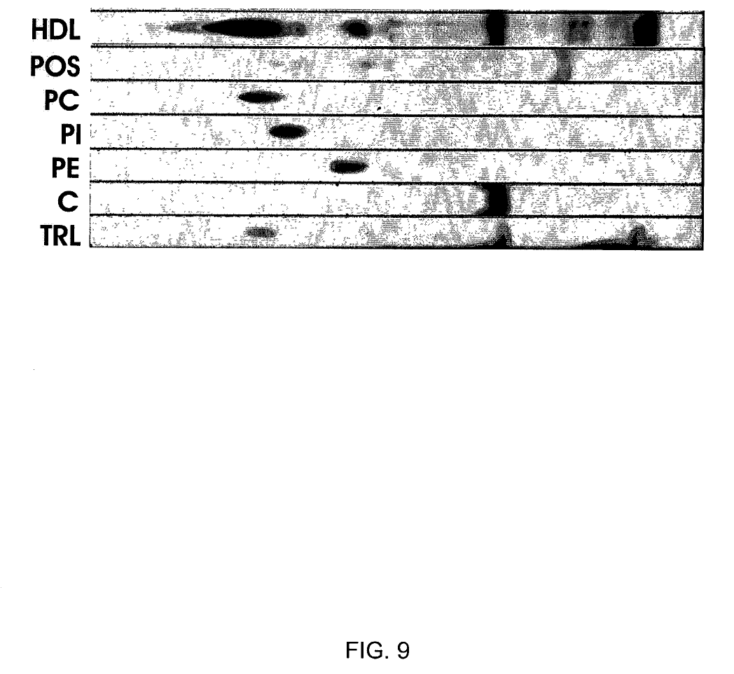

To characterize the lipids in the lipoprotein fraction, thin layer chromatography was performed. Acid charring was used to identify lipid containing spots. The spots were scraped off of the plate and ¹⁴C was quantified by liquid scintillation counting. Six of 17 ¹⁴C-containing spots were identified with standards shown (FIG. 9). Eleven ¹⁴C-containing spots bound to HDL remain unidentified and could be unique serum marker(s) for patients with early AMD.

Thus, in an embodiment of the present invention, a patient sample is obtained, such as by drawing blood, and the HDL is examined for bound POS residues. From this, a determination of their risk of visual loss from AMD is made. In a specific embodiment, the profile of bound POS residues is indicative of identifying an individual afflicted with ocular disease and/or of identifying an individual at risk for developing an ocular disease.

### Example 5

### Modulation of RCT by Compound Administration

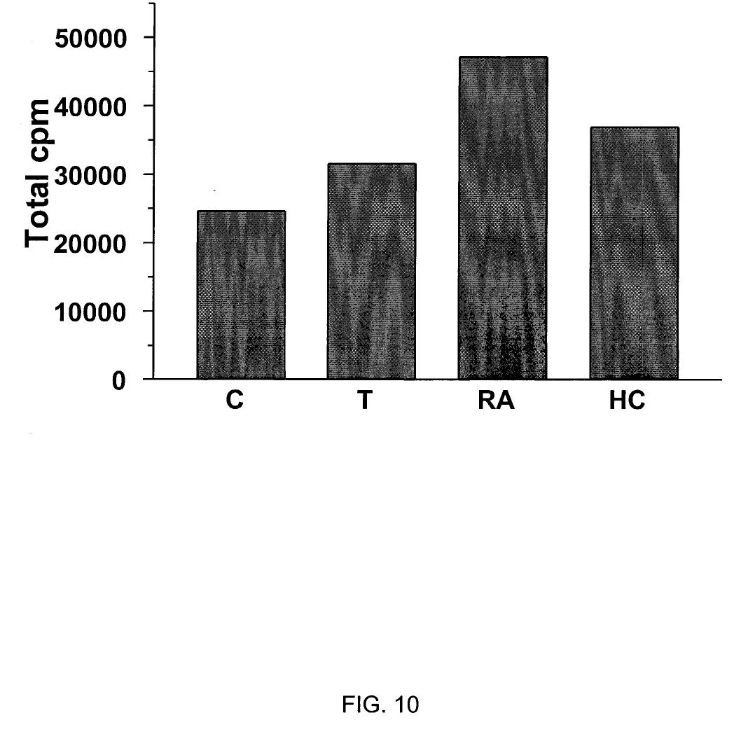

This experiment determines whether compound administration can upregulate efflux of labeled POS residues to HDL, particularly by showing regulation of ¹⁴C-DHA labeled POS efflux into basal media. An assay similar to that described in Example 4 is utilized; however, in this Example the cells were treated with T3, 9 cis-retinoic acid, and 22 (R) hydroxycholesterol in the concentrations described above for 36 hours. Total radioactivity (cpm) in the absence of HDL purification was determined by liquid scintillation counting of the basal media. FIG. 10 indicates that compound treatments increase RCT by cultured human RPE cells.

Specifically, cells were grown for 1 to 2 weeks at confluence on Transwell plates. ¹⁴C-labeled POS (30 mg/ml) were added to the apical medium. The apical and basal medium comprised either 10⁻⁷ M T3, 2.5×10⁻⁵ M 22 (R) hydroxycholesterol, or 10⁻⁷ M cis retinoic acid. The basal medium contained 1 mg/ml HDL. After 36 hours the basal medium was collected and ¹⁴C counts were determined by scintillation counting. As stated, all of the compound treatments increased transport of ¹⁴C-labeled POS to the basal medium.

The effect of T3 on Apo E mRNA levels was also assessed by RT-PCR. Treatment with 10⁻⁷ M T3 resulted in a 1.5 to 2-fold increase in apo E mRNA levels, suggesting that T3 is acting, at least in part, to increase apo E levels at the mRNA level. In specific embodiments, administration of 9 cis-retinoic acid and 22 (R) hydroxycholesterol similarly upregulates expression of apo E.

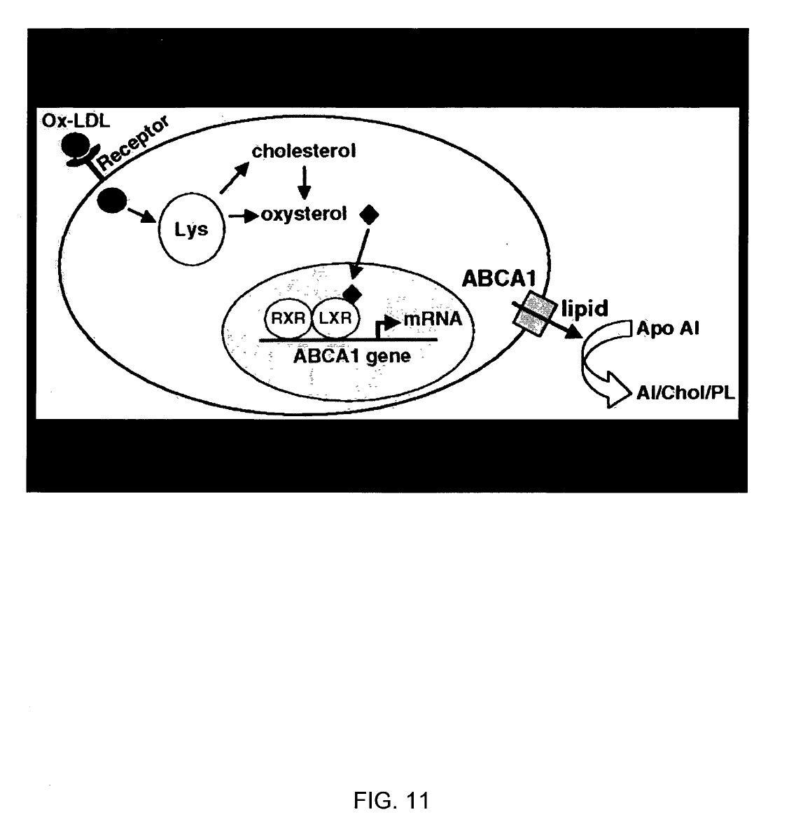

Thus, in a specific embodiment, RCT is regulated via nuclear hormone receptor ligands. For example, ABCA1 expression is upregulated by binding of LXR and RXR agonists to their respective nuclear hormone receptors (FIG. 11). Since these receptors form heterodimers bound to the ABCA1 promoter, ligand binding increases expression of ABCA1 and, hence, RCT.

### Example 6

### Identification of HDL as Lipid Acceptor from RPE Cells

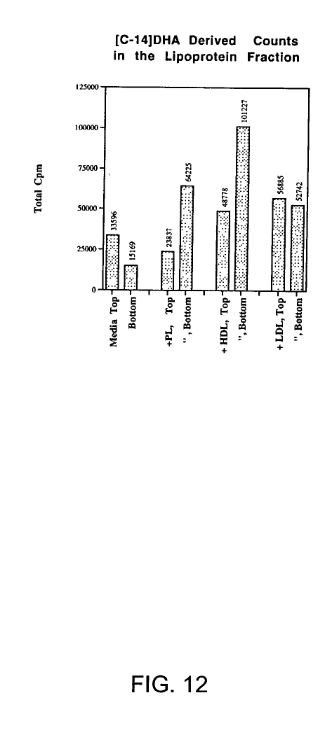

In the presence of added purified human LDL and HDL, radiolabeled lipid efflux is enhanced (FIG. 12). As shown graphically, efflux (bottoms in graph) was greatly enhanced by the presence of plasma (PL in graph), HDL or LDL, as compared to no addition to the bottom medium (left side of graph).

As shown in FIG. 8, when whole human EDTA-plasma is employed and lipoproteins are isolated, [¹⁴C]-labeled lipids are incorporated into LDL and HDL. However, radiolabel preferentially associated with HDL. Furthermore, the radiolabel in HDL was localized to the larger HDL 2 subspecies, which include the HDL particles enriched in apo E. This result suggests that lipid efflux from RPE is enhanced by the apo E-containing HDL.

### Example 7

### Reduction of BM Lipids Via Scavenger Receptors (SRS)

Scavenger receptors in macrophages function to phagocytose oxLDL molecules. There are types of SRs previously described in macrophages including SR-A1, SR-A2, SR-B1, SR-B2, CD36, and LOX. SRs are distinct from LDL receptors in that levels of expression for SRs are upregulated by oxLDL. This upregulation by intracellular oxLDL levels is modulated by nuclear hormone receptors, peroxisome proliferator activated receptor (PPAR) and retinoic acid X receptor (RXR), that exert transcriptional control of CD36 expression. Because the earliest lesion of AS, the fatty streak, consists of macrophages engulfed with excessive oxLDL, and because RPE cells similarly become filled with lipid inclusions in AMD, SR expression was studied in RPE cells. Expression of the following SRs in RPE cells was identified: CD36 (confirmation of previous investigators), SR-A1, SR-A2 (both first time demonstrated in RPE), SR-B1, SR-B2 (both first time demonstrated in RPE).

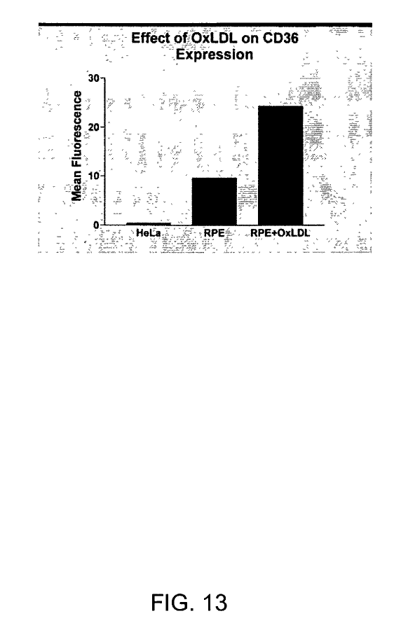

The inventors have also shown that, like macrophages, oxLDL upregulates expression of CD36 in RPE cells (FIG. 13). Additionally, RPE cells express the nuclear hormone receptors, PPAR and RXR, indicating control mechanisms for SR expression are analogous between the cell types. Thus, in specific embodiments the expression of RPE SRs in patients is controlled with PPAR and RXR ligands (e.g. PG-J2, thiazolidinediones, cis-retinoic acid). This controls the rate at which RPE cells phagocytose oxidized photoreceptor outer segments, and hence slows the rate at which abnormal lipid inclusions accumulate in RPE and BM. In other specific embodiments, expression of CD36 is downregulated with a composition such as tamoxifen, TGF-beta or INF-gamma. Similarly, regulating expression of other RPE SRs would control levels of lipids in both RPE and BM. For example, for SR-A regulation IGF-1, TGF-beta, EGF, and/or PDGF is used, and for SR-B regulation cAMP and/or estradiol (for upregulation) or TNF-alpha, LPS, and/or INF-gamma (for downregulation) is used.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference. The references, to the extent that they provide exemplary procedural or other details supplementary to those set forth herein, are specifically incorporated herein by reference.

### Patents

U.S. Pat. No. 6,071,924

U.S. Pat. No. 5,846,711

WO 00/52479

WO 01/58494

WO 02/13812

### Publications

Anderson D H, Ozaki S, Nealon M, Neitz J, Mullins R F, Hageman G S, Johnson L V: Local cellular sources of apolipoprotein E in the human retina and retinal pigmented epithelium: implications for the process of drusen formation.:Am J Ophthalmol. 2001;131(6):767-8

Anderson, D. H. et al. (2001) Local Cellular Sources of Apolipoprotein E in the Human Retina and Retinal Pigmented Epithelium: Implications for the Process of Drusen Formation, Amer. J. Ophthalm. 131(6):767-781.

Bellosta S, Mahley R, Sanan D, Murata J, Newland D, Taylor J, Pitas R. Macrophage-specific expression of human apolipoprotein E reduces atherosclerosis in hypercholesterolemic E-null mice. J Clin Invest. 1995;96:2170-217

Browning P J, Roberts D D, Zabrenetzky V, Bryant J, Kaplan M, Washington R H, Panet A, Gallo R C, Vogel T: Apolipoprotein E (ApoE), a novel heparin-binding protein inhibits the development of Kaposi's sarcoma-like lesions in BALB/c nu/nu mice. J Exp Med. 1994; 180(5):1949-54

Curcio, C. A., K. Bradley, C. Guidry, M. Kirk, L. Wilson, S. Bames, H. S. Kruth, C. C. Y. Chang, T. Y. Chang: A Local Source for Esterified Cholesterol (EC) in Human Bruch's Membrane (BrM), ARVO Abstracts 2002 (need ref)

Curcio, Christine A., Millican, C. Leigh, Bailey, Tammy, Kruth, Howard S. Accumulation of Cholesterol with Age in Human Bruch's Membrane. Invest. Ophthalmol. Vis. Sci. 2001 42: 265-274

Dithmar, Stefan, Curcio, Christine A., Le, Ngoc-Anh, Brown, Stephanie, Grossniklaus, Hans E.:Ultrastructural Changes in Bruch's Membrane of Apolipoprotein E-Deficient Mice Invest. Ophthalmol. Vis. Sci. 2000 41: 2035-2042

Duncan K G, Bailey K R, Baxter J D, Schwartz D M. The human fetal retinal pigment epithelium: A target tissue for thyroid hormones. Ophthalmic Res. 1999; 31(6):399-406.

Feeney-Burns L, Hilderbrand E S, Eldridge S: Aging human RPE: morphometric analysis of macular, equatorial, and peripheral cells. Invest Ophthalmol Vis Sci 1984; 25: 195-200.

Friedman, E. (2000) The Role of the Atherosclerotic Process in the Pathogenesis of Age-related Macular Degeneration, Amer. J. Ophthalm. 130(5):658-663.

Hasty, A. H., M. F. Linton, S. J. Brandt, V. R. Babaev, L. A. Gleaves, and S. Fazio. 1999. Retroviral gene therapy in ApoE-deficient mice: ApoE expression in the artery wall reduces early foam cell lesion formation. Circulation. 99: 2571-2576

Holz F G, Sheraidah G, Pauleikhoff D, Bird A C: Analysis of lipid deposits extracted from human macular and peripheral Bruch's membrane. Arch Ophthalmol 1994; 112: 402-406.

Janowski B A, Grogan M J, Jones S A, Wisely G B, Kliewer S A, COrey E J, Mangelsdorf D J: Structural requirements of ligands for the oxyserol liver X receptors LXRa and LXRb. Proc Natl Acad Sci (USA) 1999 Jan 96:266-271.

Kennedy C J, Rakoczy P E, Constable I J: Lipofuscin of the retinal pigment epithelium: a review. Eye 1995; 9:262-274.

Klaver C C, Kliffen M, van Duijn C M, Hofman A, Cruts M, Grobbee D E, van Broeckhoven C, de Jong P T: Genetic association of apolipoprotein E with age-related macular degeneration. Am J Hum Genet. 1998 Jul; 63(1):200-6

Kliffen, Mike, Lutgens, Esther, Daemen, Mat J A P, de Muinck, Ebo D, Mooy, Cornelia M, de Jong, Paulus T V M: The APO*E3-Leiden mouse as an animal model for basal laminar deposit Br J Ophthalmol 2000 84: 1415-1419

Laffitte B A, Repa J J, Joseph S B, Wilpitz D C, Kast H R, Mangelsdorf D J, Tontonoz P: LXRs control lipid-inducible expression of the apolipoprotein E gene in macrophages and adipocytes. Proc Natl Acad Sci U S A Jan. 16, 2001;98(2):507-12

Laffitte B A, Repa J J, Joseph S B, Wilpitz D C, Kast H R, Mangelsdorf D J, Tontonoz P:The modulation of apolipoprotein E gene expression by 3,3′-5-triiodothyronine in HepG2 cells occurs at transcriptional and post-transcriptional levels. Eur J Biochem. Sep. 1, 1994;224(2):463-71.

Langer C, Huang Y, Cullen P, Wiesenhütter B, Mahley R W, Assmann G, von Eckardstein A. Endogenous apolipoprotein E modulates cholesterol efflux and cholesterol ester hydrolysis mediated by high-density lipoprotein-3 and lipid-free apolipoproteins in mouse peritoneal macrophages. J Mol Med. 2000;78:217-227

Lin, C. Y., H. W. Duan, and T. Mazzone. 1999. Apolipoprotein E-dependent cholesterol efflux from macrophages: kinetic study and divergent mechanisms for endogenous versus exogenous apolipoprotein E. J. Lipid Res. 40: 1618-1626

Mak P A, Laffitte B A, Desrumaux C, Joseph S B, Curtiss L K, Mangelsdorf D J, Tontonoz P, Edwards P A: Regulated expression of the apolipoprotein E/C-I/C-IV/C-II gene cluster in murine and human macrophages. A critical role for nuclear liver X receptors alpha and beta. J Biol Chem. Aug. 30, 2002;277(35):31900-8

Mazzone T, Reardon C. Expression of heterologous human apolipoprotein E by J774 macrophages enhances cholesterol efflux to HDL3. J Lipid Res. 1994;35:1345-1353

Mazzone, T., L. Pusteinikas, and C. Reardon. 1992. Secretion of apoE by macrophages is accompanied by enhanced cholesterol efflux. Circulation. 86 (Suppl. I): I-2

Michael E. Kelly, Moira A. Clay, Meenakshi J. Mistry, Hsiu-Mei Hsieh-Li and Judith A. K. Harmony: Apolipoprotein E Inhibition of Proliferation of Mitogen-Activated T Lymphocytes: Production of Interleukin 2 with Reduced Biological Activity, Cellular Immunology, Volume 159, Issue 2, December 1994, Pages 124-139.

Moore D J, Clover G M: The effect of age on the macromolecular permeability of human Bruch's membrane. Invest Ophthalmol Vis Sci 2001; 42: 2970-2975

Moore D J, Hussain A A, Marshall J: Age-related variation in the hydraulic conductivity of Bruch's membrane. Invest Ophthalmol Vis Sci 1995; 36: 1290-1297.

Mullins R F, Russell S R, Anderson D H, Hageman G S. Drusen associated with aging and age-related macular degeneration contain proteins common to extracellular deposits associated with atherosclerosis, elastosis, amyloidosis, and dense deposit disease. FASEB J. 2000 May;14(7):835-46.

Pauleikhoff D, Harper C A, Marshall J, Bird A C: Aging changes in Bruch's membrane. A histochemical and morphologic study. Ophthalmology 1990; 97: 171-178.

Pauleikhoff D, Harper C A, Marshall J, Bird A C: Aging changes in Bruch's membrane. A histochemical and morphologic study. Ophthalmology 1990; 97: 171-8, 1990.

Sergio Fazio, Vladimir R. Babaev, Alisa B. Murray, Alyssa H. Hasty, Kathy J. Carter, Linda A. Gleaves, James B. Atkinson, and MacRae F. Linton: Increased atherosclerosis in mice reconstituted with apolipoprotein E null macrophages PNAS 94: 4647-4652

Sheraidah G, Steinmetz R, Maguire J, Pauleikhoff D, Marshall J, Bird A C: Correlation between lipids extracted from Bruch's membrane and age. Ophthalmology 1993; 100:47-51.

Shimano, H., J. Ohsuga, M. Shimada, Y. Namba, T. Gotoda, K. Harada, M. Katsuki, Y. Yazaki, and N. Yamada. 1995. Inhibition of diet-induced atheroma formation in transgenic mice expressing apolipoprotein E in the arterial wall. J. Clin. Invest. 95: 469-476

Simonelli F, Margaglione M, Testa F, Cappucci G, Manitto M P, Brancato R, Rinaldi E: Apolipoprotein E Polymorphisms in Age-Related Macular Degeneration in an Italian Population. Ophthalmic Res 2001;33:325-328

Souied E H, Benlian P, Amouyel P, Feingold J, Lagarde J P, Munnich A, Kaplan J, Coscas G, Soubrane G.: The epsilon4 allele of the apolipoprotein E gene as a potential protective factor for exudative age-related macular degeneration. Am J Ophthalmol. 1998;125(3):353-9

Spaide R F, Ho-Spaide W C, Browne R W, Armstrong D: Characterization of peroxidized lipids in Bruch's membrane. Retina 1999; 19: 141-147.

Starita C, Hussain A A, Pagliarini S, Marshall J: Hydrodynamics of ageing Bruch's membrane: implications for macular disease. Exp Eye Res 1996; 62: 565-572.

Tangirala R K, Pratico D, FitzGerald G A, Chun S, Tsukamoto K, Maugeais C, Usher D C, Pure E, Rader D J: Reduction of isoprostanes and regression of advanced atherosclerosis by apolipoprotein E. J Biol Chem. Jan. 5, 2001;276(1):261-6

Taylor, Hugh R, Keeffe, Jill E: World blindness: a 21st century perspective. Br J Ophthalmol 2001 85: 261-266

VanNewkirk, Mylan R., Nanjan, Mukesh B., Wang, Jie Jin, Mitchell, Paul, Taylor, Hugh R., McCarty, Cathy A.: The prevalence of age-related maculopathy: The visual impairment project Ophthalmology 2000 107: 1593-1600

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of increasing lipid efflux from an ocular tissue, comprising the step of delivering to the tissue a nuclear hormone receptor ligand.

2. The method of claim 1, wherein said ocular tissue is retinal pigment epithelium (RPE), Bruch's membrane, or both.

3. The method of claim 1, wherein said nuclear hormone receptor is thyroid hormone receptor.

4. The method of claim 3, wherein said ligand of thyroid hormone receptor is 3,5,3′-L-triiodothyronine (T3), TRIAC (3-triiodothyroacetic acid), GC-1, KB141, 3,5 dimethyl-3-isopropylthyronine, or a mixture thereof.

5. The method of claim 1, wherein said nuclear hormone receptor is liver X receptor.

6. The method of claim 5, wherein said ligand of liver X receptor is 22 (R) hydroxycholesterol, acetyl-podocarpic dimer, T0901317, GW3965 (12), 24(S),25-epoxycholesterol, 24(R),25-epoxycholesterol, 22(R)-ol-24(S),25-epoxycholesterol, 22(S)-ol,24(R),25-epoxycholesterol, 24(S),25-iminocholesterol, methyl-H-cholenate, dimethyl-hydroxycholenamide, 24(S)-hydroxycholesterol, 24(R)-hydroxycholesterol, 22(S)-hydroxycholesterol, 22(R),24(S)-dihydroxycholesterol, 25-hydroxycholesterol, 24(S),25-dihydroxycholesterol, 24(R),25-dihydroxycholesterol, 24,25-dehydrocholesterol, 7(α)-ol,24(S),25-epoxycholesterol, 7(β)-ol,24(S),25-epoxycholesterol, 7k,24(S),25-epoxycholesterol, 7(α)-hydroxycholesterol, 7-ketocholesterol, cholesterol, 5,6-24(S),25-diepoxycholesterol, or a mixture thereof.

7. The method of claim 1, wherein said nuclear hormone receptor is retinoid X receptor.

8. The method of claim 7, wherein said ligand of retinoid X receptor is 9 cis-retinoic acid, AGN 191659 [(E)-5-[2-(5,6,7,8-tetrahydro-3,5,5,8,8-pentamethyl-2-naphthyl)propen-1-yl]-2-thiophenecarboxylic acid], AGN 191701 [(E) 2-[2-(5,6,7,8-tetrahydro-3,5,5,8,8-pentamethyl-2-naphthyl)propen-1-yl]-4-thiophene-carboxylic acid], AGN 192849 [(3,5,5,8,8,-pentamethyl-5,6,7,8-tetrahydronaphthalen-2-yl) (5 carboxypyrid-2-yl)sulfide], LGD346, LG100268, LG100754, BMS649, bexaroteneR (4-[1-(5,6,7,8-tetrahydro-3,5,5,8,8-pentamethyl-2-naphthalenyl) ethenyl] benzoic acid), or a mixture thereof.

9. The method of claim 1, wherein said ocular tissue is comprised in an individual.

10. The method of claim 9, wherein said individual is afflicted with macular degeneration.

11. The method of claim 10, wherein said macular degeneration is age-related macular degeneration.

12. The method of claim 9, wherein said individual is afflicted with Stargardts disease (fundus flavimaculatus).

13. A method of increasing reverse cholesterol transport in an ocular tissue, comprising the step of delivering to the tissue at least one ligand of a nuclear hormone receptor.

14. The method of claim 13, wherein said ocular tissue is retinal pigment epithelium (RPE), Bruch's membrane, or a combination thereof.

15. The method of claim 13, wherein said nuclear hormone receptor is thyroid hormone receptor.

16. The method of claim 15, wherein said ligand of thyroid hormone receptor is T3, TRIAC (3-triiodothyroacetic acid), GC-1, K1141, 3,5 dimethyl-3-isopropylthyronine, or a mixture thereof.

17. The method of claim 13, wherein said nuclear hormone receptor is liver X receptor.

18. The method of claim 17, wherein said ligand of liver X receptor is 22 (R) hydroxycholesterol, acetyl-podocarpic dimer, T0901317, GW3965 (12), 24(S),25-epoxycholesterol, 24(R),25-epoxycholesterol, 22(R)-ol-24(S),25-epoxycholesterol, 22(S)-ol,24(R),25-epoxycholesterol, 24(S),25-iminocholesterol, methyl-H-cholenate, dimethyl-hydroxycholenamide, 24(S)-hydroxycholesterol, 24(R)-hydroxycholesterol, 22(S)-hydroxycholesterol, 22(R),24(S)-dihydroxycholesterol, 25-hydroxycholesterol, 24(S),25-dihydroxycholesterol, 24(R),25-dihydroxycholesterol, 24,25-dehydrocholesterol, 7(α)-ol,24(S),25-epoxycholesterol, 7β)-ol,24(S),25-epoxycholesterol, 7k,24(S),25-epoxycholesterol, 7(α)-hydroxycholesterol, 7-ketocholesterol, cholesterol, 5,6-24(S),25-diepoxycholesterol, or a mixture thereof.

19. The method of claim 13, wherein said nuclear hormone receptor is retinoid X receptor.

20. The method of claim 19, wherein said ligand of retinoid X receptor is 9 cis-retinoic acid, AGN 191659 [(E)-5-[2-(5,6,7,8-tetrahydro-3,5,5,8,8-pentamethyl-2-naphthyl)propen-1-yl]-2-thiophenecarboxylic acid], AGN 191701 [(E) 2-[2-(5,6,7,8-tetrahydro-3,5,5,8,8-pentamethyl-2-naphthyl)propen-1-yl]-4-thiophene-carboxylic acid], AGN 192849 [(3,5,5,8,8,-pentamethyl-5,6,7,8-tetrahydronaphthalen-2-yl) (5 carboxypyrid-2-yl)sulfide], LGD346, LG100268, LG100754, BMS649, bexaroteneR (4-[1-(5,6,7,8-tetrahydro-3,5,5,8,8-pentamethyl-2-naphthalenyl) ethenyl] benzoic acid), or a mixture thereof.

21. The method of claim 13, wherein said ocular tissue is comprised in an individual.

22. The method of claim 21, wherein said individual is afflicted with macular degeneration.

23. The method of claim 22, wherein said macular degeneration is age-related macular degeneration.

24. The method of claim 21, wherein said individual is afflicted with Stargardts disease (fundus flavimaculatus).

25. A method of treating macular degeneration (AMD) in an individual, comprising the step of delivering to the individual a ligand of a nuclear hormone receptor.

26. The method of claim 25, wherein said delivering occurs under conditions wherein reverse cholesterol transport is upregulated.

27. The method of claim 25, wherein said nuclear hormone receptor is thyroid hormone receptor.

28. The method of claim 27, wherein said ligand of thyroid hormone receptor is T3, TRIAC (3-triiodothyroacetic acid), GC-1, KB 141, 3,5 dimethyl-3-isopropylthyronine, or a mixture thereof.

29. The method of claim 25, wherein said nuclear hormone receptor is liver X receptor.

30. The method of claim 29, wherein said ligand of liver X receptor is 22 (R) hydroxycholesterol, acetyl-podocarpic dimer, T0901317, GW3965 (12), 24(S),25-epoxycholesterol, 24(R),25-epoxycholesterol, 22(R)-ol-24(S),25-epoxycholesterol, 22(S)-ol,24(R),25-epoxycholesterol, 24(S),25-iminocholesterol, methyl-H-cholenate, dimethyl-hydroxycholenamide, 24(S)-hydroxycholesterol, 24(R)-hydroxycholesterol, 22(S)-hydroxycholesterol, 22(R),24(S)-dihydroxycholesterol, 25-hydroxycholesterol, 24(S),25-dihydroxycholesterol, 24(R),25-dihydroxycholesterol, 24,25-dehydrocholesterol, 7(α)-ol,24(S),25-epoxycholesterol, 7β)-ol,24(S),25-epoxycholesterol, 7k,24(S),25-epoxycholesterol, 7(α-hydroxycholesterol, 7-ketocholesterol, cholesterol, 5,6-24(S),25-diepoxycholesterol, or a mixture thereof.

31. The method of claim 25, wherein said nuclear hormone receptor is retinoid X receptor.

32. The method of claim 31, wherein said ligand of retinoid X receptor is 9 cis-retinoic acid, AGN 191659 [(E)-5-[2-(5,6,7,8-tetrahydro-3,5,5,8,8-pentamethyl-2-naphthyl)propen-1-yl]-2-thiophenecarboxylic acid], AGN 191701 [(E) 2-[2-(5,6,7,8-tetrahydro-3,5,5,8,8-pentamethyl-2-naphthyl)propen-1-yl]-4-thiophene-carboxylic acid], AGN 192849 [(3,5,5,8,8,-pentamethyl-5,6,7,8-tetrahydronaphthalen-2-yl) (5 carboxypyrid-2-yl)sulfide], LGD346, LG100268, LG100754, BMS649, bexaroteneR (4-[1-(5,6,7,8-tetrahydro-3,5,5,8,8-pentamethyl-2-naphthalenyl) ethenyl] benzoic acid), or a mixture thereof.

33. The method of claim 25, wherein said macular degeneration is age-related macular degeneration.

34. A kit for the treatment of macular degeneration, housed in a suitable container, comprising a ligand of a nuclear hormone receptor.

35. The kit of claim 34, wherein said nuclear hormone receptor is TR, RXR, LXR, or a combination thereof.

36. The kit of claim 34, wherein said ligand is T3, 9-cis retinoic acid, (R) hydroxycholesterol, or a mixture thereof.

37. The kit of claim 34, wherein said ligand is TRIAC (3-triiodothyroacetic acid, GC-1, KB141, 3,5 dimethyl-3-isopropylthyronine, acetyl-podocarpic dimer, T0901317, GW3965 (12), 24(S),25-epoxycholesterol, 24(R),25-epoxycholesterol, 22(R)-ol-24(S),25-epoxycholesterol, 22(S)-ol,24(R),25-epoxycholesterol, 24(S),25-iminocholesterol, methyl-H-cholenate, dimethyl-hydroxycholenamide, 24(S)-hydroxycholesterol, 24(R)-hydroxycholesterol, 22(S)-hydroxycholesterol, 22(R),24(S)-dihydroxycholesterol, 25-hydroxycholesterol, 24(S),25-dihydroxycholesterol, 24(R),25-dihydroxycholesterol, 24,25-dehydrocholesterol, 7(α)-ol,24(S),25-epoxycholesterol, 7(β)-ol,24(S),25-epoxycholesterol, 7k,24(S),25-epoxycholesterol, 7(α)-hydroxycholesterol, 7-ketocholesterol, cholesterol, 5,6-24(S),25-diepoxycholesterol, AGN 191659 [(E)-5-[2-(5,6,7,8-tetrahydro-3,5,5,8,8-pentamethyl-2-naphthyl)propen-1-yl]-2-thiophenecarboxylic acid], AGN 191701 [(E) 2-[2-(5,6,7,8-tetrahydro-3,5,5,8,8-pentamethyl-2-naphthyl)propen-1-yl]-4-thiophene-carboxylic acid], AGN 192849 [(3,5,5,8,8,-pentamethyl-5,6,7,8-tetrahydronaphthalen-2-yl) (5 carboxypyrid-2-yl)sulfide], LGD346, LG100268, LG100754, BMS649, bexaroteneR (4-[1-(5,6,7,8-tetrahydro-3,5,5,8,8-pentamethyl-2-naphthalenyl) ethenyl] benzoic acid), or a mixture thereof.

38. The kit of claim 34, wherein said kit comprises a pharmaceutically acceptable excipient.

39. A method of treating macular degeneration (AMD) in an individual, comprising the steps of:
identifying a ligand for a nuclear hormone receptor; and
delivering said ligand to said individual.


SEQUENCE LISTING (12 sequences)

SEQ ID NO: 1 - DNA, 1156 bp, Human
cgcagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 2 - DNA, 5515 bp, Human
ggaacttgatgctcagagaggacaagtcatttgcccaaggtcacacagctggcaactggcagacgagattcacgccctggcaatttgactccagaatcctaaccttaacccagaagcacggcttcaagccctggaaaccacaatacctgtggcagccagggggaggtgctggaatctcatttcacatgtggggagggggctcctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctgtattactgggcgaggtgtcctcccttcctggggactgtggggggtggtcaaaagacctctatgccccacctccttcctccctctgccctgctgtgcctggggcagggggagaacagcccacctcgtgactgggctgcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaagccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcattatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttgggggcgctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcagacagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggccggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtagctagatgcctggacggggtcagaaggaccctgacccgaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagcaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctcccccgcctccccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactgccacacaattctcagccccctcctctccatctgtgtctgtgtgtatctttctctctgccctttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctggctcaagagatcctccgccatcggcctcccaaagtgctgggattccaggcatgggctccgagcggcctgcccaacttaataatattgttcctagagttgcactc

SEQ ID NO: 3 - DNA, 1157 bp, Human
ccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaagtcacccaagaactgagggcgctgatggacgagaccatgaaggagttgaaggcctacaaatcggaactggaggaacaactgaccccggtagcggaggagacgcgggcacggctgtccaaggagctgcagacggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatcccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagtcggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 4 - protein, 317 aa, Human [sequence omitted]

SEQ ID NO: 5 - protein, 317 aa, Human [sequence omitted]

SEQ ID NO: 6 - protein, 317 aa, Human
MKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 7 - DNA, 10412 bp, Human
gtaattgcgagcgagagtgagtggggccgggacccgcagagccgagccgacccttctctcccgggctgcggcagggcagggcggggagctccgcgcaccaacagagccggttctcagggcgctttgctccttgttttttccccggttctgttttctccccttctccggaaggcttgtcaaggggtaggagaaagagacgcaaacacaaaagtggaaaacagttaatgaccagccacggcgtccctgctgtgagctctggccgctgccttccagggctcccgagccacacgctgggggtgctggctgagggaacatggcttgttggcctcagctgaggttgctgctgtggaagaacctcactttcagaagaagacaaacatgtcagctgctgctggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccaaaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccagaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgtctgtgttttcttcttgtggttgtatatatcaggtaaaatattttccaaagagccatgtgtcatgtaatactgaaccactttgatattgagacattaatttgtacccttgttattatctactagtaataatgtaatactgtagaaatattgctctaattcttttcaaaattgttgcatcccccttagaatgtttctatttccataaggatttaggtatgctattatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagatttctaagttaaatcattttcattaaaaggaaaaaagaaaaaaaattttgtatgtcaataactttatatgaagtattaaaatgcatatttctatgttgtaatataatgagtcacaaaataaagctgtgacagttctgttggtctacagaaa

SEQ ID NO: 8 - DNA, 6786 bp, Human
atggcttgttggcctcagctgaggttgctgctgtggaagaacctcactttcagaagaagacaaacatgtcagctgctgctggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccaaaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagagtgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatga

SEQ ID NO: 9 - protein, 2261 aa, Human
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 10 - protein, 2261 aa, Human; identical to SEQ ID NO: 9

SEQ ID NO: 11 - DNA, 2751 bp, Human
ggcacgaggccggatccaccgtgcctctgcggcctgcgtgcccggagtccccgcctgtgtcgtctctgtcgccgtccccgtctcctgccaggcgcggagccctgcgagccgcgggtgggccccaggcgcgcagacatgggctgctccgccaaagcgcgctgggctgccggggcgctgggcgtcgcggggctactgtgcgctgtgctgggcgctgtcatgatcgtgatggtgccgtcgctcatcaagcagcaggtccttaagaacgtgcgcatcgaccccagtagcctgtccttcaacatgtggaaggagatccctatccccttctatctctccgtctacttctttgacgtcatgaaccccagcgagatcctgaagggcgagaagccgcaggtgcgggagcgcgggccctacgtgtacagggagttcaggcacaaaagcaacatcaccttcaacaacaacgacaccgtgtccttcctcgagtaccgcaccttccagttccagccctccaagtcccacggctcggagagcgactacatcgtcatgcccaacatcctggtcttgggtgcggcggtgatgatggagaataagcccatgaccctgaagctcatcatgaccttggcattcaccaccctcggcgaacgtgccttcatgaaccgcactgtgggtgagatcatgtggggctacaaggacccccttgtgaatctcatcaacaagtactttccaggcatgttccccttcaaggacaagttcggattatttgctgagctcaacaactccgactctgggctcttcacggtgttcacgggggtccagaacatcagcaggatccacctcgtggacaagtggaacgggctgagcaaggttgacttctggcattccgatcagtgcaacatgatcaatggaacttctgggcaaatgtggccgcccttcatgactcctgagtcctcgctggagttctacagcccggaggcctgccgatccatgaagctaatgtacaaggagtcaggggtgtttgaaggcatccccacctatcgcttcgtggctcccaaaaccctgtttgccaacgggtccatctacccacccaacgaaggcttctgcccgtgcctggagtctggaattcagaacgtcagcacctgcaggttcagtgcccccttgtttctctcccatcctcacttcctcaacgccgacccggttctggcagaagcggtgactggcctgcaccctaaccaggaggcacactccttgttcctggacatccacccggtcacgggaatccccatgaactgctctgtgaaactgcagctgagcctctacatgaaatctgtcgcaggcattggacaaactgggaagattgagcctgtggtcctgccgctgctctggtttgcagagagcggggccatggagggggagactcttcacacattctacactcagctggtgttgatgcccaaggtgatgcactatgcccagtacgtcctcctggcgctgggctgcgtcctgctgctggtccctgtcatctgccaaatccggagccaagtaggtgctggccagagggcagcccgggctgacagccattcgcttgcctgctgggggaaaggggcctcagatcggaccctctggccaaccgcagcctggagcccacctccagcagcagtcctgcgtctctgccggagtgggagcggtcactgctgggggctgcgcagcacgcttgcgtcttttgcatgccgcgttgccactactctgcctgttctggaaggcctgggaccctcccttggagggggcacagggtcctgaggacaccgtgagccagccaggcctggccgctgggcctgaccggccccccagcccctacaccccgcttctcccggactctcccagcggacagccccccagccccacagcctgagcctcccagctgccatgtgcctgttgcacacctgcacacacgccctggcacacatacacacatgcgtgcaggcttgtgcagacactcagggatggagctgctgctgaagggacttgtagggagaggctcgtcaacaagcactgttctggaaccttctctccacgtggcccacaggcctgaccacaggggctgtgggtcctgcgtccccttcctcgggtgagcctggcctgtcccgttcagccgttgggcccaggcttcctcccctccaaggtgaaacactgcagtcccggtgtggtggctccccatgcaggacgggccaggctgggagtgccgccttcctgtgccaaattcagtggggactcagtgcccaggccctggccacgagctttggccttggtctacctgccaggccaggcaaagcgcctttacacaggcctcggaaaacaatggagtgagcacaagatgccctgtgcagctgcccgagggtctccgcccaccccggccggactttgatccccccgaagtcttcacaggcactgcatcgggttgtctggcgcccttttcctccagcctaaactgacatcatcctatggactgagccggccactctctggccgaagtggccgcaggctgtgcccccgagctgcccccaccccctcacagggtccctcagattataggtgcccaggctgaggtgaagaggcctgggggccctgccttccgggcgctcctggaccctggggcaaacctgtgacccttttctactggaatagaaatgagttttatcatctttgaaaaataattcactcttgaagtaataaacgtttaaaaaaaaaaaaaaaaaa

SEQ ID NO: 12 - protein, 552 aa, Human
MGCSAKARWAAGALGVAGLLCAVLGAVMIVMVPSLIKQQVLKNVRIDPSSLSFNMWKEIPIPFYLSVYFFDVMNPSEILKGEKPQVRERGPYVYREFRHKSNITFNNNDTVSFLEYRTFQFQPSKSHGSESDYIVMPNILVLGAAVMMENKPMTLKLIMTLAFTTLGERAFMNRTVGEIMWGYKDPLVNLINKYFPGMFPFKDKFGLFAELNNSDSGLFTVFTGVQNISRIHLVDKWNGLSKVDFWHSDQCNMINGTSGQMWPPFMTPESSLEFYSPEACRSMKLMYKESGVFEGIPTYRFVAPKTLFANGSIYPPNEGFCPCLESGIQNVSTCRFSAPLFLSHPHFLNADPVLAEAVTGLHPNQEAHSLFLDIHPVTGIPMNCSVKLQLSLYMKSVAGIGQTGKIEPVVLPLLWFAESGAMEGETLHTFYTQLVLMPKVMHYAQYVLLALGCVLLLVPVICQIRSQVGAGQRAARADSHSLACWGKGASDRTLWPTAAWSPPPAAVLRLCRSGSGHCWGLRSTLASFACRVATTLPVLEGLGPSLGGGTGS